(19) United States
(12) Patent Application Publication
(10) Pub. No.: US 2004/0266790 A1
(43) Pub. Date: Dec. 30, 2004

(54) Risperidone monohydrochloride

(76) Inventors: Jiri Bartl, Strelice (CZ); Reinerus G. Gieling, Nijmegen (NL)

Correspondence Address: MARK R. BUSCHER, P.O. BOX 161, CATHARPIN, VA, 20143, US

(21) Appl. No.: 10/825,683

(22) Filed: Apr. 16, 2004

Related U.S. Application Data
(60) Provisional application No. 60/464,364, filed on Apr. 22, 2003.

(51) Int. Cl. A61K 31/519; C07D 487/04

(52) U.S. Cl. 514/259.41; 544/279

(57) ABSTRACT

Monohydrochloride salts of risperidone have been found to have useful properties. A preferred form is crystalline risperidone monohydrochloride hemipentahydrate. The monohydrochloride salts can be used in pharmaceutical compositions and methods such as for use in treating psychotic disorders.

This application claims the benefit from U.S. Provisional Application Ser. No. 60/464,364, filed Apr. 22, 2003, the entire contents of which are incorporated herein by reference.

### BACKGROUND OF THE INVENTION

The present invention relates to monohydrochloride salts of risperidone and the use thereof as a pharmaceutical active agent.

Risperidone, or 3-[2-[4-(6-Fluoro-1,2-benzisoxazol-3-yl)-1-piperidinyl]-ethyl]-6,7,8,9-tetrahydro-2-methyl-4-H-pyrido[1,2-a]-pyrimidin-4-one, is a serotonin antagonist approved for the treatment of psychotic disorders such as schizophrenia. Its structure is shown in formula (1).

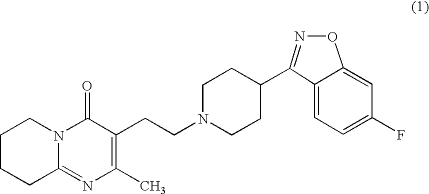

Risperidone is approved for marketing in the U.S.A. under the name RISPERDAL by Janssen, as a free base in both tablet and oral solution dosage forms. Risperidone base is only sparingly soluble in water (approximately 4 mg/ml).

The compound and its pharmaceutical activity are identified in U.S. Pat. No. 4,804,663 as one of several compounds in a class of 3-piperidinyl-1,2-benzisoxazoles or -1,2-benzisothiazoles. Although pharmaceutically acceptable acid addition salts of the entire class of compounds disclosed in U.S. Pat. No. 4,804,663 are taught as being useful, the examples therein synthesize and pharmaceutically test only the free base form of the compounds.

U.S. Pat. No. 5,453,425 and U.S. Pat. No. 5,616,587 disclose stable aqueous solutions of risperidone for oral or parenteral administration. Apparently, the generic solution formulations disclosed in EP 0 196 132, which corresponds to U.S. Pat. No. 4,804,663, provide unsatisfactory stability when risperidone is used as the active ingredient. Both of these patents, U.S. Pat. No. 5,453,425 and U.S. Pat. No. 5,616,587, disclose the use of a buffer to maintain the pH of the aqueous solution within the range of 2 to 6. The solution is taught to be essentially free of sorbitol. The buffer system is described as a mixture of appropriate amounts of an acid and a base. A tartaric acid/sodium hydroxide buffer system is preferred. The solution is taught to be formed by, inter alia, dissolving the acid component of the buffer and the risperidone into heated water; stirring until complete dissolution; and then cooling the solution and adding the base component of the buffer to adjust the pH. The solution can be further diluted with water to a final end-volume.

U.S. Pat. No. 5,616,587 further explains that the tartaric acid/sodium hydroxide buffer system is preferred in part because risperidone tartrate has good aqueous solubility and further reports that risperidone tartrate has a room temperature solubility of about 80 mg/ml while risperidone hydrochloride has a room temperature solubility of about 19.6 mg/ml. However, no description is set forth on how the salt was formed, whether it was formed as a solid and/or isolated form, or on how the solubility test was made. Indeed, given how the solution is formed, it would appear that the salt was formed in situ, i.e. in a dissolved state, and the solubility limit determined from the maximum amount of risperidone base that could be dissolved into the solution. In any event and regardless of such speculation, the patent does not disclose obtaining a solid form of a risperidone salt.

It would be advantageous to provide a pharmaceutically suitable risperidone salt form. It would be further advantageous to provide a stable solid state salt form.

### SUMMARY OF THE INVENTION

The present invention relates to the discovery and isolation of monohydrochloride salts of risperidone. Accordingly, a first aspect of the invention relates to a monohydrochloride salt of risperidone. The invention includes a variety of salt forms including dissolved, liquid/oil, and solid forms, especially crystalline forms including hydrate and anhydrate forms. A preferred salt is crystalline risperidone monohydrochloride hemipentahydrate, although crystalline risperidone monohydrochloride anhydrates are also useful crystalline forms.

Another aspect of the invention relates to a pharmaceutical composition comprising a monohydrochloride salt of risperidone and at least one pharmaceutically acceptable excipient. The composition includes unit dosage forms especially solid oral dosage forms (i.e. tablets, capsules, etc.) as well as liquid forms including oral liquids. In a unit dosage form the composition typically contains an effective anti-psychotic amount of risperidone monohydrochloride. Generally the amount corresponds to 0.1 to 20 mg of the risperidone base. The monohydrochloride salt of risperidone well tolerates the presence of sorbitol in a liquid composition and thus compositions containing sorbitol are a further embodiment of the invention.

Another aspect of the present invention relates to a process for making a monohydrochloride salt of risperidone, which comprises contacting a risperidone donor with a chloride ion donor in a solvent; and optionally precipitating a crystalline risperidone monohydrochloride salt. In a preferred process, the risperidone donor is a monovalent salt of risperidone, e.g., formed from an acid other than hydrochloric acid, especially acetic acid, and the chloride ion donor is a chloride salt, especially sodium chloride. Such a preferred process can be carried out in water as a solvent.

A further aspect of the invention relates to a method of treating a psychotic disorder in a mammal which comprises administering an effective amount of a monohydrochloride salt of risperidone to a mammal in need thereof.

### BRIEF DESCRIPTION OF THE DRAWINGS

FIG. 1. is a DSC scan of a crystalline risperidone monohydrochloride anhydrate.

FIG. 2. is an X-ray powder diffraction pattern corresponding to a risperidone monohydrochloride anhydrate.

FIG. 3. is a DSC scan of a crystalline risperidone monohydrochloride hemipentahydrate.

FIG. 4. is an X-ray powder diffraction pattern corresponding to crystalline risperidone monohydrochloride hemipentahydrate.

FIG. 5 is an X-ray powder diffraction pattern corresponding to a de-hydrated crystalline risperidone monohydrochloride hemipentahydrate.

FIG. 6. is an X-ray powder diffraction pattern corresponding to crystalline risperidone monohydrochloride hemipentahydrate.

### DETAILED DESCRIPTION OF THE INVENTION

The present invention relates to the discovery of a new salt of risperidone, namely the monohydrochloride salt. A “salt” of risperidone means a mixture of ionic risperidone and a counter-ion(s). In a crystalline state, the ions have a fixed spatial relationship forming, optionally with water or solvent, a crystal lattice. In a dissolved state, however, the dissolved ions may either have some degree of association or the ions can be completely dissociated.

In a risperidone salt, the ratio of risperidone ion to counter-ion can vary depending generally upon the counter-ion and the method of formation. This is because risperidone has more than one nitrogen atom that is susceptible to protonation and thus it can have more than one counter ion. Hence, risperidone may form various types of salts with the same acid. Concerning a hydrochloride acid addition salt, risperidone may form a monohydrochloride or a dihydrochloride salt. The risperidone “monohydrochloride” is any hydrochloride salt of risperidone that comprises essentially a 1:1 molar ratio of risperidone and chloride moieties. The risperidone moiety is thus protonated on one nitrogen atom to have one positive charge while the chloride counter-ion has one off-setting negative charge. Because the nitrogen atoms in risperidone are not equally susceptible to protonation, or salt formation, a monohydrochloride salt is believed to involve protonation of the most susceptible nitrogen atom without the other nitrogen atoms being protonated. While theoretically the ratio of risperidone base to chloride ion is exactly 1:1, typically the measured values can have a variation up to 0.2, more typically not greater than 0.1. This variation can be due to measuring error as well as impurities including small amounts of risperidone base and/or risperidone dihydrochloride.

The monohydrochloride salts of risperidone generally have, in an essentially pure form, a water solubility in terms of the risperidone base of around 7.5 mg/ml+/−0.5 mg/ml. Impurities, especially a dihydrochloride salt of risperidone can increase the solubility in as much as the water solubility of the dihydrochloride salt is greater than 80 mg/ml. Thus the presence of the dihydrochloride salt in small amounts can increase the water solubility of a monohydrochloride salt form. The monohydrochloride salt of risperidone according to the present invention preferably has a water solubility of 10 mg/ml or less, more preferably 5-9 mg/ml, and still more preferably 6-8 mg/ml, each expressed in terms of the amount of free base of risperidone. The solubility is normally determined by forming a saturated solution in equilibrium for 24 hours and measuring the amount of risperidone present in the supernatant by HPLC or other suitable analytical means.

Correspondingly, a monohydrochloride salt of risperidone that is substantially free from a dihydrochloride salt of risperidone is a preferred embodiment of the invention, especially with regard to a dissolved salt but also in solid state. In this embodiment the amount of dihydrochloride salt, in terms of moles, should be less than 5%, preferably less than 2%, and more preferably less than 1%, based on the total amount of risperidone salt.

The risperidone monohydrochloride can be obtained in solid or dissolved form. In solid form it is preferably crystalline, although amorphous or non-crystalline forms are also contemplated. In crystalline form it is preferably obtained in isolated form; i.e. substantially separated from unbound solvent, such as by filtration and/or drying, etc., and substantially free from other compounds such as synthetic precursors and/or side products. The solid state salt, whether isolated or not, preferably has a purity of at least 70%, more typically at least 90%, more preferably at least 95%, still more preferably at least 98%, and most preferably at least 99%, wherein the percentages are based on weight. If intended for use in a pharmaceutical dosage composition, the risperidone salt typically has a purity of at least 99.8% including 99.9%.

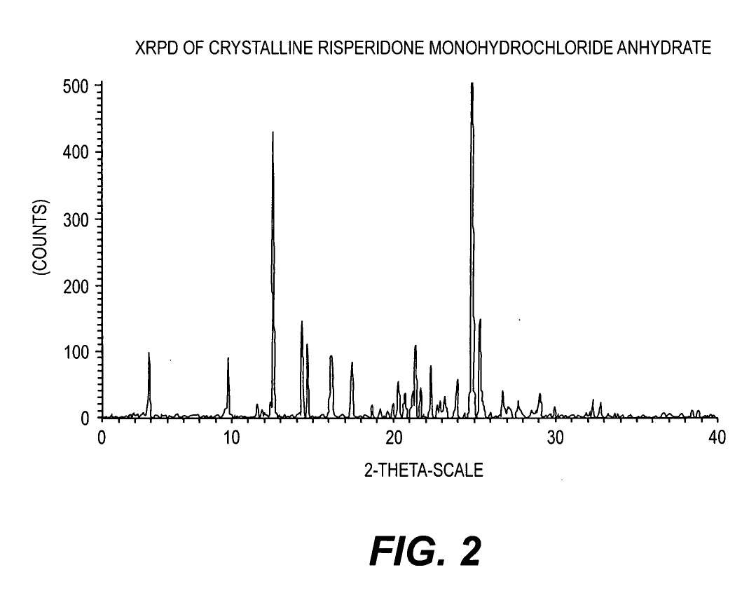

Crystalline risperidone monohydrochloride can be formed in hydrated or anhydrated forms. An anhydrate means that no water is present as part of the repeating lattice structure. In practice, however, an anhydrate may contain some water, such as adsorbed to the surface of the material and/or from insufficient drying. Accordingly, an anhydrate should contain not more than 1% water, weight, preferably not more than 0.8%, more preferably not more than 0.5%. A preferred anhydrate form substantially corresponds to the X-ray powder diffraction pattern shown in FIG. 2. This anhydrate form is stable and non-hygroscopic. Other anhydrate forms are also possible, including a de-hydrated crystalline form as discussed hereinafter.

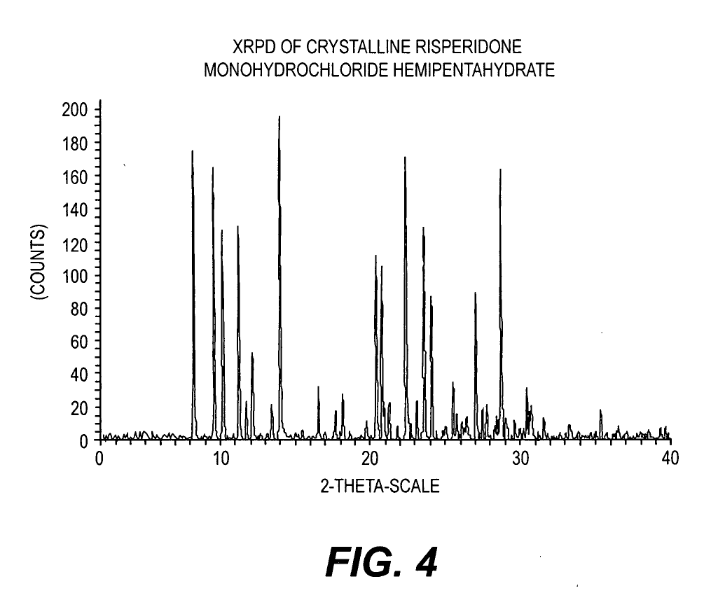

Hydrates are any crystal that contains water as part of the repeating unit or cell that forms the crystal lattice. Typically the amount of bound water in the hydrate is at least 1.0% by weight, more preferably at least 1.5%. As a hydrate, the most preferred is crystalline risperidone monohydrochloride hemipentahydrate. The term “hemipentahydrate” is used to denote that the crystal contains about two and half moles of water per each mole of risperidone. In terms of percent, the water content in a dry product is about 7-9.5%, preferably 7.5-9.5%, more preferably 8.5-9.5% and especially 8.6-9.2%, based on the total weight of the risperidone hydrochloride material. As shown by X-ray analysis, the crystalline lattice of risperidone monohydrochloride hemipentahydrate comprises repeating units or cells consisting of two molecules of risperidone monohydrochloride and five molecules of water, in a fixed spatial relationship. The X-ray powder diffraction pattern for a representative crystalline risperidone monohydrochloride hemipentahydrate is shown in FIG. 4. Correspondingly a preferred embodiment of the present invention is a risperidone monohydrochloride salt that exhibits an X-ray powder diffraction pattern that substantially corresponds to FIG. 4.

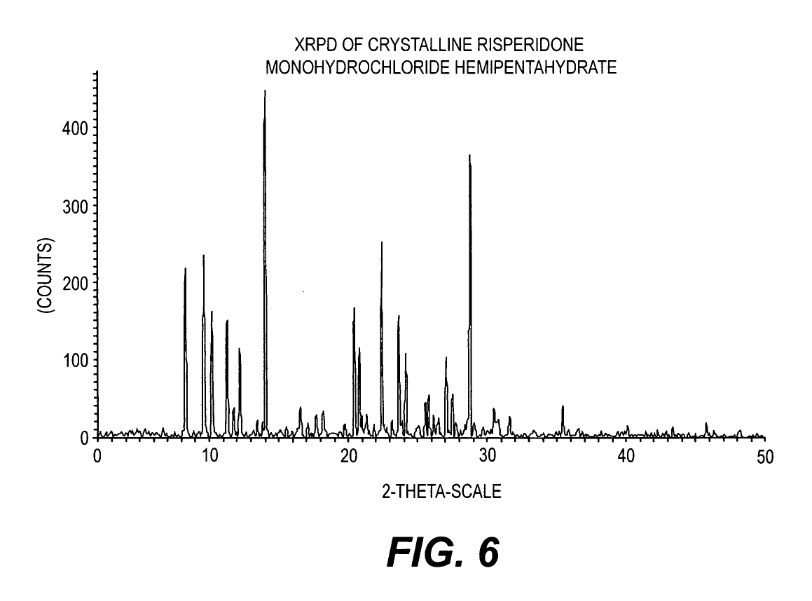

The phrase “substantially corresponds” in the context of an X-ray powder diffraction pattern is used to allow for variations caused by different sample preparations, different equipment and/or settings used in measuring, normal experimental error/variation and small amounts of impurities. Differences in a pattern that are not attributable to these factors indicate that the pattern in question does not substantially correspond to the pattern of FIG. 2 or 4. For example, the pattern for the example 11 material, shown in FIG. 6, substantially corresponds to the pattern in FIG. 4, even though it is not an identical, superimposable image. As is readily apparent to workers skilled in the art of comparing X-ray powder diffraction patterns, FIGS. 4 and 6 show that the underlying materials have the “same” crystalline structure.

The hemipentahydrate form (hereinafter sometimes abbreviated “hph”) can be obtained in block and/or cube shaped crystals which are advantageous for handling and/or formulating into solid dosage forms such as tablets. The anhydrous form tends to be formed in needle shaped crystals that are less desirable; i.e. needles are inferior to blocks in terms of compressibility, etc. The water solubility is essentially the same for both hydrates and anhydrates and the pH of a saturated solution is physiologically suitable for making pharmaceutical formulations including parenteral or oral liquids, even without the use of a buffer or pH adjusting agent.

The risperidone hydrochloride hph is relatively stable and non-hygroscopic. After 2 weeks exposure at 40° C./75% RH, no water uptake was observed. However, it is possible to dehydrate the crystal, usually reversibly but also irreversibly. In general, heating up to 40° C. does not dehydrate the crystal; i.e. no bound water loss. However, heating at 60° C., for example, or at 40° C. under a vacuum, can remove some or almost all of the crystal water from the lattice. However, the water loss is reversible by simple exposure to air and generally quickly such as a few hours to a few days. In contrast, heating at 80° C. for two days produces a water content of about 0.5% which after subsequent exposure to 40° C./75% RH for five days further drops to 0.1%. This thermally dehydrated hph crystal is stable, non-hygroscopic and apparently substantially corresponds to the anhydrous form of FIG. 2. A similar transition can be induced by heating at 75° C. as illustrated by Example 6B hereinafter. It was observed however, that simply heating at 80° C. does not automatically convert the hph crystal to an irreversible anhydrate crystal form. For example, heating at 80° C. for only 1.5 hours can reduce the water content to 1.2% (a hydrate form), and upon subsequent exposure to hot and humid conditions (40° C./75% RH) water is slowly reacquired and it is believed that eventually the original water content of the hph crystal is obtained. In summary, the hph crystal is stable under drying conditions of up to at least 40° C. without vacuum. At higher temperatures or with vacuum, the hph crystal may loose some or all of its bound water but will readily reacquire the water upon exposure to ambient conditions. And at significant thermal stress such as 75° C. or higher for two or more days the hph crystal can be thermally altered into a stable non-hygroscopic anhydrate form.

The crystalline risperidone monohydrochloride hph is also well soluble in ethanol and interestingly, in mixtures of ethanol and water. For example at 24° C., the risperidone monohydrochloride hph has the following solubility in various ethanol mixtures:

| Ethanol:water (v/v) | mg/ml |
|---|---|
| 3:1 | 60 |
| 1:1 | 60 |
| 1:3 | 25 |
| 1:6 | 16 |

Risperidone monohydrochloride according to the present invention can be made in general by a salt forming reaction or a salt exchange reaction. Thus, a risperidone donor is contacted with a chloride ion donor in a solvent under suitable conditions to form a risperidone monohydrochloride salt. A risperidone donor is any molecule or complex that can provide risperidone and is generally risperidone free base and/or a monovalent risperidone salt other than risperidone monohydrochloride. In some embodiments the risperidone donor is preferably a monovalent salt formed from a weak acid, i.e. an acid that does not fully dissociate when dissolved in water, such as risperidone acetate. Weak acids such as acetic acid are advantageous in that the monovalent salt can be exclusively formed; i.e. the weak acid can be of insufficient strength to protonate the second nitrogen atom in the risperidone compound. Such a risperidone donor is especially useful in a salt or ion exchange reaction. The risperidone donor can be in isolated form or contained within a synthesis product or mixture. The chloride ion donor is any molecule that provides a chloride ion for the reaction including hydrochloric acid or a salt derived from hydrochloric acid. Preferably the salt is a water soluble inorganic chloride salt such as sodium chloride, potassium chloride, calcium chloride, or ammonium chloride.

The contacting of the two donors occurs in solvent. However, it should be understood that such does not require both donors to be fully dissolved in the solvent, although such a condition can be preferable. For example, a slurry or suspension of the risperidone donor wherein the liquid phase contains the chloride ion donor provides contact of the two donors in the liquid phase, i.e. in the solvent, to form the monohydrochloride salt of risperidone.

The “solvent” can be a single liquid or a mixture of two or more and thus the term “solvent” embraces the singular as well as the plural forms of the word; i.e. solvents. The solvent facilitates the contacting of the risperidone and chloride ion donors and generally at least one of the risperidone donor and the chloride ion donor is soluble therein. Suitable solvents include water, a lower aliphatic alcohol, a lower aliphatic ketone such as acetone, an ether such as diethylether or tetrahydrofuran, a hydrocarbon such as hexane, and mixtures thereof. Preferably the solvent contains water, a lower aliphatic alcohol (C₁-C₄ alcohols), most preferably ethanol, or a mixture thereof. In some embodiments, the solvent preferably contains no water or only a minor amount of water, i.e. up to 50% water, preferably up to 20% water, more preferably 3 to 10% (v/v). In other embodiments, the solvent is mostly or all water, especially at least 80% water, more preferably at least 90% water, and typically essentially 100% water (v/v). The other solvent, if any, is a water miscible solvent such as a lower aliphatic alcohol, preferably ethanol.

The two donors are contacted by any suitable technique. While a two-phase system is possible, such as a slurry or an organic-water liquid system, preferably a single phase is used. The order and rate of contacting the solution comprising the risperidone donor with the chloride ion donor can vary. Advantageously, the chloride ion donor, used as such or dissolved in a solvent, especially an aqueous solvent, is added at once, portionwise or continually, to a stirred solution or suspension of risperidone donor. The order of contacting may also be reversed.

If the solubility of the risperidone donor or the chloride ion donor in the solvent is found to be insufficient for the intended purpose, it may be enhanced by common means, e.g. by heating the mixture (optionally up to reflux) or adding a co-solvent to enhance the solubility. Preferably, the concentration of risperidone donor, the kind of solvent, and the temperature of contact are so selected that a clear solution is, at least temporarily, formed. In any event and regardless of whether slurries or suspensions are employed of the risperidone donor or chloride ion donor, the salt forming reaction occurs in a dissolved state. The temperature of the solvent during the contact can be constant or variable and is not particularly limited. Typically the solvent temperature is from 5° C. to the reflux temperature of the solvent, more typically from room temperature (20° C.) up to the reflux temperature of the solvent.

Normally it is desirable to precipitate the resulting risperidone monohydrochloride from the solution. In a preferred case, the risperidone monohydrochloride precipitates from the solution spontaneously due to a difference in solubility between the formed salt and the starting materials in the solvent. Optionally, the precipitation may be induced by a suitable conventional technique(s), or the yield of precipitation may be enhanced by such technique(s). The techniques preferably comprise, alone or in combination:

a) cooling the reaction mixture, including spontaneous cooling, i.e. without applying a cooling device, of a previously heated solution;

b) concentrating the reaction mixture including essentially evaporating/removing the whole amount of the solvent;

c) adding a contrasolvent wherein the contrasolvent—a liquid in which the formed salt is less soluble—may be miscible or immiscible with the solvent; and/or d) adding a seed crystal at anytime during the process including from before contacting to after precipitation has begun.

The general process for making a monohydrochloride salt of risperidone can be sub-divided into two general preferred embodiments. In a first embodiment, the chloride ion donor is hydrochloric acid, generally in the form of an aqueous solution. The risperidone donor can be a free base or an acid addition salt derived from an acid that is less strong than hydrochloric acid. The hydrochloric acid should be combined with the risperidone donor in no more than a slight molar excess, e.g. 1.1:1 of HCl:risperidone or less, in order to avoid the formation of the dihydrochloride salt.

A risperidone monohydrochloride anhydrate can be conveniently formed by mixing in ethanol a risperidone base with one equivalent of aqueous hydrochloric acid and precipitating the resulting monohydrochloride salt. The risperidone solution is normally heated in order to dissolve all the risperidone base. After addition of the aqueous hydrochloric acid, precipitation of the anhydrous monohydrochloride salt of risperidone from the solution is generally easily obtained by allowing the solution to cool. Precipitation can occur, for example, upon cooling down to 65° C. or less. The presence of water does not force the formation of a hydrate form. And advantageously, the presence of water suppresses the precipitation of the dihydrochloride salt. Highly concentrated hydrochloric acid solutions, e.g. 6 Molar or higher, tend to provide too little water such that the more highly water soluble dihydrochloride salt is precipitated along with the lower water soluble monohydrochloride salt. Less concentrated solutions, which require more volume in order to provide the same amount of hydrochloric acid, provide more water to the system and thus help insure that any dihydrochloride salt formed remains in solution and separate from the crystallized monohydrochloride salt. Typically a concentration of 1-4 Molar is preferred. The total water in such a solvent, after addition, is generally 20% or less, more typically 10% or less, although higher amounts can be used. The above technique is a preferred method for forming the crystalline risperidone monohydrochloride anhydrate having the x-ray diffraction pattern substantially corresponding to FIG. 2.

Risperidone hydrochloride hemipentahydrate may be produced by this method as well, provided however that the solvent essentially comprises water. As the process, however, has a big risk of over-protonation of risperidone and thus forming the dihydrochloride salt, which reduces the yield, the second embodiment described below is a more preferred process.

The second embodiment uses a chloride salt as the chloride ion donor. In this embodiment the risperidone donor is preferably a salt of risperidone, especially a weak acid addition salt of risperidone. Contacting of the chloride salt with the risperidone salt can affect a salt or ion exchange whereby the counter ion of the risperidone is replaced with hydrochloride. This method has several advantages. By using a chloride salt, such as NaCl, the risk of forming the dihydrochloride salt of risperidone is significantly reduced. Whereas using excess hydrochloric acid (i.e., more than 20% excess) increases the probability of dihydrochloride production, the chloride salt can be used in great excess without significantly increasing the probability of forming the dihydrochloride risperidone salt. Advantageously the risperidone donor is a water soluble monovalent salt such as risperidone acetate or risperidone mesylate. In this way, (1) the already protonated nitrogen is far more likely to react than the unprotonated nitrogens which leads to monohydrochloride salt formation and (2) the reaction/contacting can use water as the solvent. By using a water soluble salt as the risperidone donor, the less soluble risperidone monohydrochloride readily precipitates from the aqueous solution. Also, the water solvent insures that the solid, precipitated risperidone monohydrochloride salt does not contain any dihydrochloride risperidone salt as an impurity due to its significantly greater water solubility. That is, should any dihydrochloride salt be formed, it would be unlikely to precipitate with the monohydrochloride salt and instead would simply remain in solution due to its much greater water solubility.

This embodiment is especially advantageous for forming hydrated forms, particularly the risperidone monohydrochloride hph salt. For example, risperidone free base can be suspended in water and acetic acid added thereto to form a solution of risperidone acetate. Then a slight molar excess of sodium chloride is added and risperidone monohydrochloride hph precipitates from the solution in high yields. Alternatively, the risperidone acetate can be formed from an organic reaction mixture which contains risperidone, such as the synthesis product mixture, by adding aqueous acetic acid thereto. The formed risperidone acetate is soluble in the water and is thus separated from the organic reaction mixture. The aqueous phase comprising the formed water soluble risperidone acetate salt solution is then used, after purification and/or filtration if necessary, for the step of contacting with the chloride ion donor. Of course other water soluble monovalent salts of risperidone can be used in this technique.

Regardless of how the risperidone monohydrochloride salt is formed, it can be isolated by conventional techniques and if needed purified to the desired degree of purity by various methods. For instance, it may be recrystallized, optionally after treatment of the solution with a suitable adsorption material, e.g. with activated charcoal. The solvents disclosed above as useful for making the monohydrochloride salts are also useful for recrystallization. For instance, suitable solvents for recrystallization are water, especially for the risperidone monohydrochloride hph, and water/ethanol mixtures especially for risperidone monohydrochloride anhydrate.

The risperidone monohydrochloride of the present invention can be formulated into various pharmaceutical compositions. The aqueous solubility of risperidone monohydrochloride is similar to risperidone free base so that the compound is a suitable alternative to the risperidone base. Contrary to the base, however, risperidone monohydrochloride is present in an ionized form; thus, the hydrophilicity of risperidone is higher, which could be an advantage.

A suitable pharmaceutical composition comprises a monohydrochloride salt of risperidone and a pharmaceutically acceptable excipient(s). Typically the composition is a finished dosage form also referred to as a unit dose. The pharmaceutical compositions of the present invention include the unit dosage forms as well as the intermediate bulk formulations such as pellets, beads, granules, powder blends, concentrated solutions, etc. Dosage forms include oral dosage forms such as tablets or oral solutions, topical dosage forms such as a transdermal patch, parenteral dosage forms such as an injectable solution, and rectal dosage forms such as a suppository, but is not limited thereto. Oral dosage forms are the most preferred due to the ease of administration and include solid oral dosage forms such as capsules, tablets, sachets/granules, and powders, as well as liquid oral dosage forms such as solutions, suspensions, and emulsions. Preferred dosage form is an oral solution, especially an aqueous solution, and a tablet, especially a rapidly disintegrating tablet.

Pharmaceutically acceptable excipients can be in solid state or liquid state as is well known in the art and include carriers, diluents, fillers, binders, lubricants, disintegrants, glidants, colorants, pigments, taste masking agents, sweeteners, plasticizers, and any acceptable auxiliary substances such as absorption enhancers, penetration enhancers, surfactants, co-surfactants, and specialized oils. The proper excipient(s) are selected based in part on the dosage form, the intended mode of administration, the intended release rate, and manufacturing reliability. Examples of common types of excipients include various polymers, waxes, calcium phosphates, sugars, and solvents. Polymers include cellulose and cellulose derivatives such as HPMC, hydroxypropyl cellulose, hydroxyethyl cellulose, microcrystalline cellulose, carboxymethylcellulose, sodium carboxymethylcellulose, calcium carboxymethylcellulose, and ethylcellulose; polyvinylpyrrolidones; polyethylenoxides; polyalkylene glycols such as polyethylene glycol and polypropylene glycol; polyacrylic acids including their copolymers and crosslinked polymers thereof, i.e. Carbopol® (B.F. Goodrich), Eudragit® (Rohm), polycarbophil, and chitosan polymers; and polyvinyl alcohols. Waxes include white beeswax, microcrystalline wax, carnauba wax, hydrogenated castor oil, glyceryl behenate, glycerylpalmito stearate, saturated polyglycolyzed glycerate. Calcium phosphates include dibasic calcium phosphate, anhydrous dibasic calcium phosphate, and tribasic calcium phosphate. Sugars include simple sugars such as lactose, maltose, mannitol, fructose, sorbitol, sacarose, xylitol, isomaltose, and glucose as well as complex sugars (polysaccharides) such as maltodextrin, amylodextrin, starches, and modified starches. Solvents are typically water or ethanol or a mixture thereof.

Solid compositions for oral administration of risperidone monohydrochloride salts may exhibit immediate or extended release of the active substance from the composition. Such compositions preferably comprise risperidone monohydrochloride hemipentahydrate and at least one solid state excipient. Solid pharmaceutical compositions are preferably formulated into tablets. The tablets may be disintegrable or monolithic. The tablets may be produced by any standard tabletting technique, e.g. by wet granulation, dry granulation or direct compression. A preferred tablet is an orally disintegrable tablet, i.e. a composition that disintegrates directly in the mouth. Various systems are known in the art and they are applicable to the compound of our invention. Preferred however is an orally disintegrating tablet comprising at least 50% silicified microcrystalline cellulose as described in commonly-owned U.S. Provisional patent application 60/463,027, filed Apr. 16, 2003, the entire contents of which are incorporated herein by reference. The silicified microcrystalline cellulose is preferably the intimate physical mixture of colloidal silicon dioxide with microcrystalline cellulose as described in U.S. Pat. No. 5,585,115. The amount of silicon dioxide is normally within the range of 0.1 to 20 wt % and more typically 1.25 to 5 wt % such as about 2 wt %. Surprisingly, such an excipient can form a tablet matrix that is orally disintegrating; i.e., the tablet disintegrates in the mouth in 80 seconds or less, preferably 2 to 50 seconds. The amount of silicified microcrystalline cellulose is preferably 50% to 90%, more preferably 60% to 80% based on the weight of the tablet.

Risperidone monohydrochloride may alternatively be blended into compositions that are suitable for being formulated into pellets. A plurality of risperidone pellets comprising the single dose of risperidone may be encapsulated into capsules made from pharmaceutically acceptable material, such as hard gelatin. In another mode, a plurality of pellets may be compressed together with suitable binders and disintegrants to form a disintegrable tablet that, upon ingestion, decomposes and releases the pellets. In yet another mode, the plurality of pellets may be filled into a sachet.

Pharmaceutical compositions comprising risperidone monohydrochloride and intended as final dosage forms for administration preferably contain a therapeutically effective amount of risperidone. The amount of the risperidone salt, expressed in terms of risperidone base, in the unit dose is usually from 0.1 to 20 mg, preferably 0.25 mg, 0.5 mg, 1 mg, 2 mg, 3 mg, 4 mg, 6 mg, or 8 mg. The unit dose in a tablet form can be one or more tablets administered at the same time. In the last case, several smaller tablets may be advantageously filled into a gelatin capsule to form a unit dose. The unit dose of a granulate or pellets in a capsule form advantageously comprises a single capsule.

Besides solid pharmaceutical compositions, risperidone monohydrochloride is also suitable for making liquid pharmaceutical compositions for oral or parenteral administration. Preferably these solutions are aqueous, meaning that water comprises a substantial portion of the solvent medium. Usually water comprises at least 50% of the solvent, preferably at least 60%, more preferably at least 80%, still more preferably at least 90%, and most preferably essentially 100% of the solvent. The remainder of the solvent may be, for instance, ethanol. In addition to containing the risperidone monohydrochloride salt as an active ingredient and a solvent, these compositions may contain auxiliary ingredients such as preservatives, tensides, isotonizing agents, flavors, colors etc.

However, it is an advantage of the risperidone monohydrochloride that the solution is not required to contain a buffering system. That is, the inventive solution preferably has only a stoichiometric or near stoichiometric amount of acid anion as opposed to a buffer system which requires a molar excess of an acid. This is possible because risperidone monohydrochloride itself exhibits, contrary to the risperidone base, the desired inherent pH for making a pharmaceutically acceptable solution. Specifically, orally administrable solutions should preferably have a pH of 3.5-8.5, while parenterally administrable solutions should preferably have a pH of 4-9. The inherent or native pH of a saturated aqueous solution of risperidone monohydrochloride is about 5, which is within both of the desired ranges. Thus, a pharmaceutical solution may be made just by dissolving the monohydrochloride salt of risperidone in water.

Furthermore, the risperidone monohydrochloride is compatible in solution with carbohydrate sweeteners such as sorbitol. This is surprising due to the earlier disclosure in WO 96/01652 that sorbitol causes instability of risperidone solutions and should be avoided from the pharmaceutical composition. In testing 1 mg/ml aqueous solutions of risperidone monohydrochloride comprising 2% sorbitol and 0.2% of benzoic acid at various stress temperatures (up to 80° C.), it was found that such solutions are surprisingly stable.

The unit dose of an injectable solution is advantageously one vial. Oral solution is preferably delivered in a multidose package, wherein the unit dose may be defined by the number of droplets, teaspoons or by means of a calibrated vial. Preferred concentration of risperidone monohydrochloride in oral or parenteral solutions is from 0.1 mg/ml to 10 mg/ml, particularly of about 1 mg/ml or 2 mg/ml, in terms of the amount of risperidone base.

The monohydrochloride salts of risperidone can be used to treat psychotic disorders including schizophrenia in animals, preferably mammals such as humans. The method comprises administering an anti-psychotic effective amount of a monohydrochloride salt of risperidone to an animal patient, preferably a mammalian patient, in need thereof. The effective amount is generally within the range of 0.001 mg/kg to 0.4 mg/kg of body weight, more preferably 0.004 mg/kg to 0.2 mg/kg of body weight, expressed with regard to the free base. Preferably the risperidone salt is administered as a unit dosage from as described above. It should be understood that a single administration includes taking one or more unit dosage forms at essentially the same time, e.g. taking two tablets.

The entire disclosure in each of the patents and journal articles mentioned in the above description is incorporated herein by reference. The invention will be further described with reference to the following non-limiting examples.

### Example 1

### Risperidone Monohydrochloride

1.98 g of risperidone was suspended in 50 ml of ethanol, affording a white suspension. 8 ml of an aqueous HCl solution (1.2 M) was added, resulting in a clear solution. After one day, a small amount of crystals had been formed. The mixture was then cooled on ice/salt, giving more crystallization. Then, the crystals were filtered off, washed with cold ethanol and dried in vacuo at 40° C., affording 1.08 g of risperidone hydrochloride. Recrystallization of the product from ethanol/water (25:1) gave pure risperidone monohydrochloride.

### Example 2

### Risperidone Monohydrochloride

1.98 g risperidone was suspended in 30 ml of ethanol, affording a white suspension and heated until a clear solution was obtained. 4 ml of an aqueous HCl solution (1.2 M; 1 equiv.) was added, and stirred for 20 h at room temperature. The mixture was then cooled to −20° C., giving rise to crystallization. Then at room temperature, the crystals were filtered off and dried in vacuo at 40° C. for 5 days, affording 198 mg risperidone monohydrochloride, mp. 275-279° C.; water content: 0.18%. Water content after exposure to air under ambient conditions for 3 weeks: 0.39%. Acid titration confirms monohydrochloride salt.

### Example 3

### Risperidone Hydrochloride Anhydrate

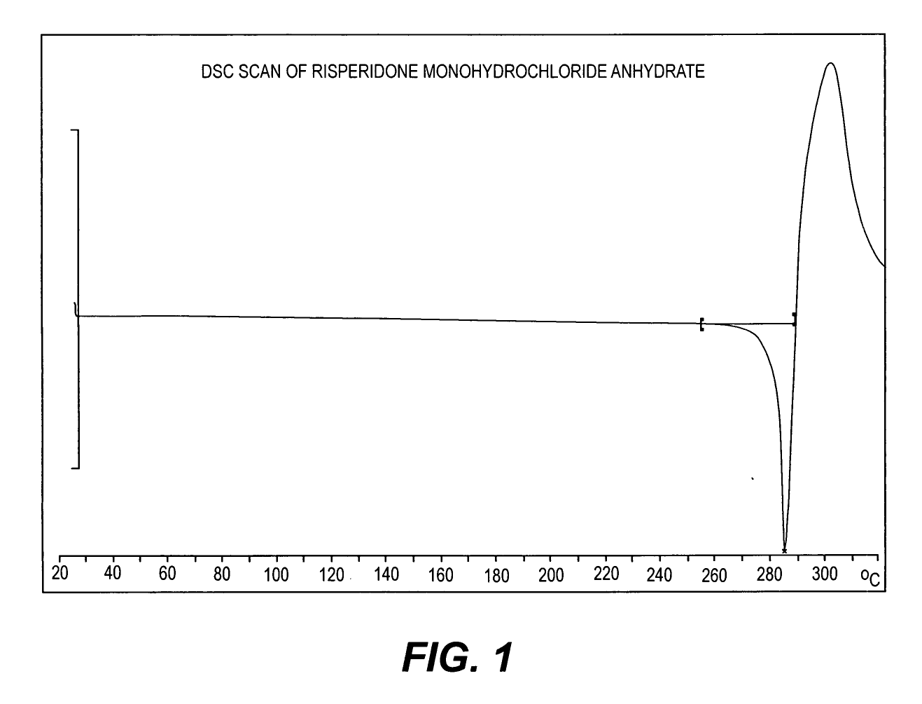

10.0 g risperidone base was dissolved in 100 ml of ethanol by heating the mixture. 8.2 ml of 3M aqueous HCl was added. After several minutes (T=55° C.), crystallization started. The resulting crystals were filtered off after 2 h, washed once with ethanol, and dried at 40° C. in vacuo for 24 h. Titration with base confirmed monohydrochloride salt; no dihydrochloride is present. Yield: 7.44 g (68%) as small white needles; water content: <0.1%. The differential scanning calorimetry (DSC) scan of this material corresponds to FIG. 1 with a melting endotherm peak at about 286° C.

### Example 4

### Risperidone Hydrochloride Anhydrate

25.0 g risperidone base was dissolved in 250 ml of ethanol by heating the mixture. 20 ml of 3M aqueous HCl was added and the mixture was allowed to cool. After several minutes (T=65° C.), crystallization started. The mixture was then further cooled to room temperature and stirred for 17 hours at room temperature. The crystals were filtered off, washed once with ethanol, and dried at 40° C. in vacuo for 24 h. Titration with base confirmed monohydrochloride salt; no dihydrochloride is present. Yield: 19.0 g as small white needles of risperidone monohydrochloride anhydrate. The powder X-ray diffraction (XRPD) pattern of this material corresponds to FIG. 2.

### Example 5

### Risperidone Hydrochloride Hemipentahydrate

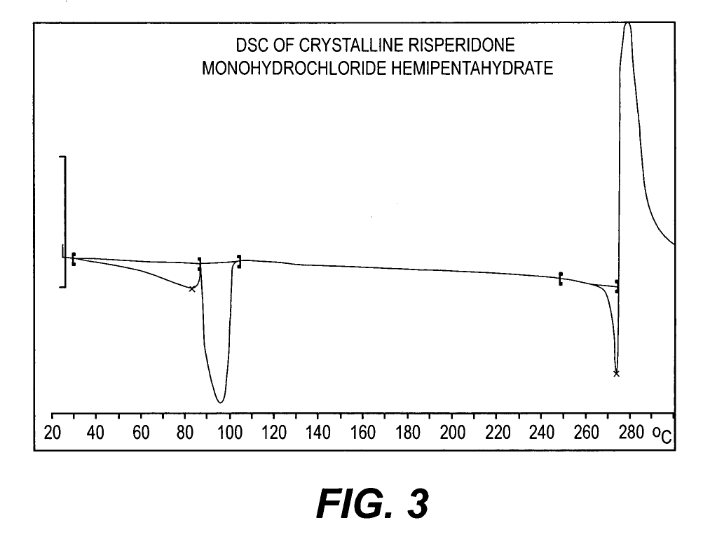

5.0 g risperidone base was stirred in 32.5 ml of water at room temperature. 1.25 ml of acetic acid was added and the mixture was stirred for 10 minutes, giving a yellowish solution, which was filtrated over a glass filter. The filter was washed twice with 2.5 ml of water. A solution of 0.85 g of sodium chloride in 4 ml of water was added to the filtrate and stirred at room temperature. After a few minutes, precipitation started. The mixture was stirred for 2 hours and then filtered off. The crystals were washed twice with 4 ml of water, and dried at room temperature for 18 hours. Yield: 5.31 g as off-white crystalline powder. Water content: 8.9%; HPLC assay 100.3%; H+titrated 100.2%; and Cl⁻ titrated 98.6%. The differential scanning calorimetry (DSC) scan of this material corresponds to FIG. 3.

### Example 6

### Risperidone Hydrochloride Hemipentahydrate

400 g of risperidone base was charged in a round-bottom flask. 800 ml of water was added and the mixture was stirred mechanically. 59 ml of acetic acid was added followed by addition of 200 ml of water to the slurry. After 45 minutes, the yellow solution was filtered over a P3 filter, which was subsequently washed with 2*300 ml water. The combined filtrates were charged in a round-bottom flask, and a solution of 68.4 g of sodium chloride in 300 ml of water was added while mechanically stirring. An off-white solid started to crystallize slowly. After 75 minutes the solid was filtered off and washed with 2*300 ml of water. The crystals were dried at room temperature for 18 hours, then at 35-40° C. for 24 hours. Yield: 455.1 g. Water content: 9.23% (2.52 equiv.), determined by both KF coulometry and loss on drying. HPLC assay 100.6%; Acetate content: 0.16 m/m %. The powder X-ray diffraction (XRPD) pattern of this material corresponds to FIG. 4.

### Example 6A

### Dehydration of Risperidone Hydrochloride Hemipentahydrate

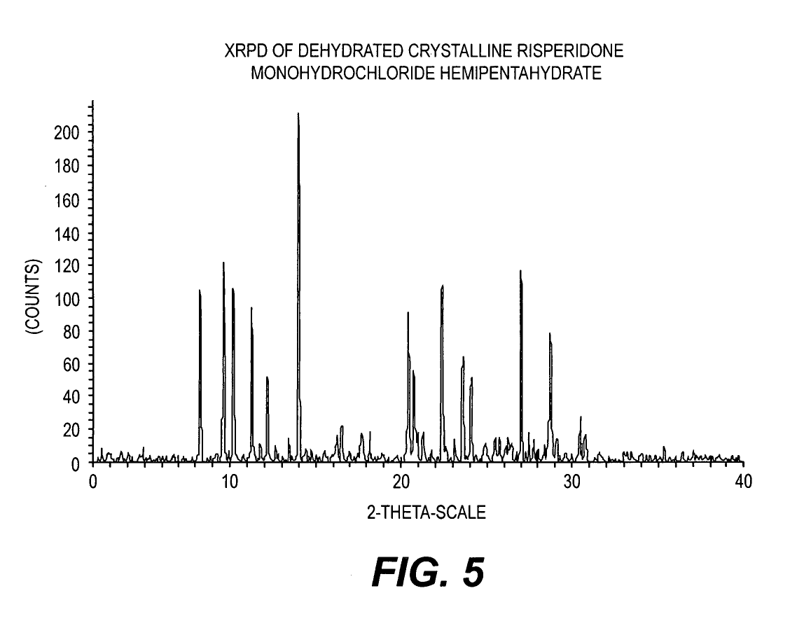

A sample of risperidone hydrochloride hemipentahydrate from the Example 6A was dried in vacuo at 40° C. for five days until the water content had decreased to 0.55%. Then X-ray powder diffraction spectrum of the anhydrated material was measured. The spectrum corresponds to FIG. 5. The XRPD-spectrum shows that at 40° C. in vacuo, risperidone monohydrochloride hemipentahydrate is dehydrated to give an anhydrated form that has substantially the same crystalline structure as the hemipentahydrate. The sample is expected (and other experiments confirmed) to re-uptake water into the crystalline structure to a water content of about 8.9%; thereby returning to the hemipentahydrate crystalline salt.

### Example 6B

### Dehydration of Risperidone Hydrochloride Hemipentahydrate

A sample of risperidone monohydrochloride hemipentahydrate from Example 6 was kept at 75.3° C. in a climate room for 64 h. At the start, a quick XRPD measurement was carried out in 24 min, showing an initial spectrum corresponding to FIG. 4; i.e. hemipentahydrate. Subsequently, every 3 hours a measurement was performed for a total of 21 measurements. The resulting spectra slowly changed from the original spectrum of risperidone monohydrochloride hemipentahydrate to a spectrum substantially similar to the anhydrate form as shown in FIG. 2. No intermediate forms were observed. Thus, over time at 75° C., risperidone monohydrochloride hemipentahydrate is dehydrated and transforms into an anhydrate crystal form, i.e. the same anhydrated form as the one that was prepared from ethanol in Example 4.

### Example 7

### Risperidone Monohydrochloride Hemipentahydrate

47.75 g of risperidone base was suspended in 300 ml of water. The suspension was acidified with 10 ml of glacial acetic acid (1.5 molar eq.). The turbid solution was stirred for 10 minutes and filtered. The filtrate was divided into two equal portions.

The first filtrate was mixed with 42.5 ml of ethanol and 5.5 ml of concentrated HCl (approximately 12N) was added dropwise. Crystals started to separate in 5-10 minutes. The suspension was stirred for 1 hour. The solid was filtered off and washed with 2×15 ml of water. Yield: 18.8 g

The second filtrate was mixed with 5.5 ml of concentrated HCl (approximately 12N) added dropwise. Crystals started to separate in 5-10 minutes. The suspension was stirred for 1 hour. The solid was filtered off and washed with 2×15 ml of water. Yield: 22.3 g.

### Example 8

### Risperidone Hydrochloride Hemipentahydrate

20.0 g of risperidone base was suspended in 40 ml of water. The suspension was acidified with 3.1 ml of acetic acid (1.1 molar eq.), stirred for 10 minutes and filtered. The filter was washed with 2×20 ml of water. Then a solution of 3.42 g of sodium chloride in 15 ml of water was prepared and filtered. The filtered solution was added into combined risperidone filtrates and the mixture was stirred at room temperature. Crystals started to separate after a while. The suspension was stirred for 1 hour; the crystals were filtered off, washed with 2×15 ml of water and dried at a temperature of 35-40° C. Yield: 22.52 g.

### Example 9

### Risperidone Hydrochloride Hemipentahydrate

23.87 g of risperidone base was suspended in 150 ml of water. The suspension was acidified with 5 ml of acetic acid. The turbid solution was stirred for 10 minutes and filtered. The filter was washed with 2×20 ml of water. 5.5 ml of concentrated HCl (approximately 12N) was added dropwise into the combined filtrate under stirring. Crystals started to separate during 1-2 minutes. The suspension was stirred for 1 hour, crystals were filtered and washed with 2×15 ml of water. Yield: 22.23 g

### EXAMPLE 10

### Risperidone Hydrochloride Hemipentahydrate from Synthesis

A mixture of 20 g of 3-[2-[4-[(2,4-difluorophenyl)-(Z-hydroxyimino)methyl]-1-piperidinyl]ethyl]-6,7,8,9-tetrahydro-2-methyl-4H-pyrido[1,2a]pyrimidin-4-one, 1.33 g of borax, a solution of 2.60 g of sodium hydroxide in 6.0 ml water, and 34 ml of ethanol was agitated at 50° C. for 1 hour. By this, a risperidone-containing reaction mixture (98% of theoretical yield of risperidone according to HPLC) was obtained. The reaction mixture was diluted with 130 ml of water and the suspension was acidified with 6.0 ml of glacial acetic acid until a solution was obtained. The solution was cooled to room temperature, filtered and the filter was washed with 25 ml of water. Then 34 ml of ethanol was distilled off from the filtrate. The solution was cooled to room temperature and a solution of 3.26 g of sodium chloride in 13 ml of water was added. The suspension was agitated for 1 hour at room temperature, the crystals were filtered off and washed with 2×15 ml of water. After drying, 21.40 g of risperidone hydrochloride hemipentahydrate was obtained (purity 99.45% by HPLC).

### Example 11

### Risperidone Hydrochloride Hemipentahydrate

0.9 ml of concentrated hydrochloric acid was dissolved in 10 ml of water and 4.1 g of risperidone base was added. The suspension was heated until the solid was dissolved (80-90° C.). The solution was allowed to cool by standing at room temperature and then in refrigerator at 5° C. for 1 hour. The solid product was filtered off and washed with 5 ml of cooled water. The product was dried on air. Yield 4.4 g of risperidone hydrochloride hemipentahydrate. The XRPD of the material corresponds to FIG. 6.

### Example 12

### Recrystallization of Risperidone Hydrochloride Hemipentahydrate

306 mg of risperidone hydrochloride hemipentahydrate was added to 30 ml of ethanol and heated to reflux, giving a clear solution. Small needles crystallized slowly, when the solution was allowed to cool to room temperature. After two hours, the crystals were filtered off and dried on the air for 17 hours. Yield: 102 mg of risperidone monohydrochloride anhydrate. Water content: 0.75%; DSC confirms anhydrate.

### Example 13

### Pharmaceutical Solutions

a) Oral solution

| Composition | (m/V %) |
|---|---|
| Risperidone HCl | 0.1% |
| | (as the base) |
| benzoic acid | 0.2% |
| saccharin | 0.1% |
| Flavors | qs |
| Water | ad 100% |

b) Parenteral solution

| Composition: | (m/V %) |
|---|---|
| Risperidone HCl | 0.2% |
| | (as the base) |
| NaCl | 0.9% |
| Na EDTA | 0.01% |
| Water | ad 100% |

Preparation of solutions:

All excipients, starting with risperidone monohydrochloride salt (in the crystalline anhydrate or hemipentahydrate form) are dissolved in 80% of the quantity of water. After everything is dissolved, the pH is checked and, optionally, NaOH and/or HCl is used to titrate the solutions to the target pH. Finally the solution is brought to its target volume with purified water, resulting in an oral solution of the risperidone concentration 1 mg/ml or a parenteral solution of 2 mg/ml. The parenteral solution is then sterilized in a suitable apparatus for an appropriate time.

### Example 14

### Pharmaceutical Oral Solution and Stability Tests

The following stock solutions were used to make three batch solutions, BS 1 through BS 3:

Stock solution 1: A solution of 1.8 mg of risperidone monohydrochloride (prepared from an anhydrate solid form) and 3.32 mg of benzoic acid (a preservative) per 1 g of water.

Stock solution 2: A solution of 167 mg sorbitol per 1 g of water.

Stock solution 3: A solution of 118 mg sorbitol per 1 g of water.

Stock solution 4: A solution of 62.5 mg sorbitol per 1 g of water.

Batch solution 1: 150.6 g of Stock solution 1, 91.1 g of Stock solution 2 and 8.3 g of water. BS 1 thus contains 0.271 g of risperidone monohydrochloride, 0.499 g of benzoic acid and 15.18 gram of sorbitol in 250 g of solution (0.1% [w/w] of risperidone, calculated as a base).

Batch solution 2: 150.6 g of Stock solution 1, 85.0 g of Stock solution 3 and 14.5 g of water. BS 2 thus contains 0.271 g of risperidone monohydrochloride, 0.499 g of benzoic acid and 10.00 gram of sorbitol in 250 g of solution (0.1% [w/w] of risperidone, calculated as a base).

Batch solution 3: 150.6 g of Stock solution 1, 80.5 g of Stock solution 4 and 18.9 g of water. BS 3 thus contains 0.271 g of risperidone monohydrochloride, 0.499 g of benzoic acid and 5.04 gram of sorbitol in 250 g of solution (0.1% [w/w] of risperidone, calculated as a base).

All the batch solutions were submitted for stability testing and the results are shown in the following table, wherein the percent impurities were determined by HPLC by internal normalization, in respect to risperidone base.

| | 40° C./75% | | |
|---|---|---|---|
| | RH | 60° C. | Acceptable |
| | 3 months | 3 months | limit |
| BS 1 | | | |
| Highest level of an identified | 0.01% | 0.05% | 0.1% |
| impurity | | | |
| Total unknown impurities | 0.03% | 0.15% | 0.5% |
| Highest unknown impurity | 0.01% | 0.06% | 0.2% |
| Total impurities | 0.05% | 0.23% | 1.0% |
| BS 2 | | | |
| Highest level of an identified | 0.01% | 0.04% | 0.1% |
| impurity | | | |
| Total unknown impurities | 0.02% | 0.12% | 0.5% |
| Highest unknown impurity | 0.01% | 0.04% | 0.2% |
| Total impurities | 0.03% | 0.18% | 1.0% |
| BS 3 | | | |
| Highest level of an identified | 0.01% | 0.02% | 0.1% |
| impurity | | | |
| Total unknown impurities | 0.02% | 0.11% | 0.5% |
| Highest unknown impurity | 0.01% | 0.04% | 0.2% |
| Total impurities | 0.03% | 0.14% | 1.0% |

All three solutions proved to be stable at 40° C./75% RH and even at 60° C. for at least 3 months. Interestingly the amount of sorbitol, up to 6% in the batch solution, does not appear to have an influence on the stability of the solutions.

### Example 15

### Pharmaceutical Oral Solution

Dissolve 2.0 gram of benzoic acid in approx. 900 ml of water, if necessary under heating. Dissolve 1.13 gram of risperidone hydrochloride hemipentahydrate and 40 gram of sorbitol in this solution at room temperature. Add water to make up total weight of solution to 1000 gram.

### EXAMPLE 16

### Orally Disintegrating Tablets

Composition per tablet:

| | |
|---|---|
| Risperidone HCl.hph | 4.0 mg |
| | (in terms of risperidone base) |
| Silicified cellulose (Prosolv HD-90) | 78.0 mg |
| L-HPC | 5.0 mg |
| Aspartame | 6.0 mg |
| Mint flavor | 6.0 mg |
| Acesulfam K | 0.5 mg |
| Sodium stearyl fumarate | 0.5 mg |

Tablets are prepared by mixing risperidone monohydrochloride hemipentahydrate, L-HPC, aspartame, mint flavor, Acesulfam K, and 30% of the Prosolv in a free fall mixer. The remaining 70% amount of Prosolv is then added and the material mixed again. The sodium stearyl fumarate is then added and the material mixed again. The mixed homogenous powder blend is compressed into 8 mm diameter round tablets having an average weight of 100 mg and an average hardness between 30 and 40 N.

### Example 17

### Capsules Comprising Risperidone Monohydrochloride Hemipentahydrate

Capsules of risperidone monohydrochloride can be made according to the following guide:

| | Strength (mg of risperidone base) | | | | | |
|---|---|---|---|---|---|---|
| | 1 | 2 | 3 | 4 | 6 | 8 |
| Risperidone HCl.hph(as | 1.0 | 2.0 | 3.0 | 4.0 | 6.0 | 8.0 |
| base) | | | | | | |
| Lactose monohydrate* | 94.7 | 94.2 | 141.3 | 188.4 | 68.4 | 91.2 |
| Microcrystalline cellulose* | 94.7 | 94.2 | 141.3 | 188.4 | 86.4 | 91.2 |
| Sodium starch glycollate | 8.0 | 8.0 | 1.2 | 1.6 | 6.0 | 8.0 |
| Colloidal silica | 0.6 | 0.6 | 0.9 | 1.2 | 0.45 | 0.6 |
| Mg stearate | 1.0 | 1.0 | 1.5 | 2.0 | 0.75 | 1.0 |
| TOTAL MASS | 200 | 200 | 300 | 400 | 150 | 200 |

The * indicates that the amount of lactose and/or microcrystalline cellulose can be adjusted to compensate for (offset) the differing weights of the risperidone monohydrochloride salts; i.e. the hph weighs more per mole than the anhydrate because of water contained therein, thereby needing less lactose and/or cellulose for the composition to equal the total targeted mass of the capsule.

Preparation of capsule composition:

Risperidone salt is mixed well with 50% of the amount of the microcrystalline cellulose (MCC), then the other 50% of the MCC is added and mixed, followed by mixing with the lactose and the sodium starch glycollate. Finally the silica is added and mixed. The entire blend is screened over a 850 micrometer sieve, and mixed again, then Mg stearate is added and mixed, resulting in a blend for capsule filling. The blend is filled into capsule size no.3 (150 mg, 200 mg), no. 1 (300 mg) or no.0 (400 mg) containing a dose of 1 mg to 8 mg of risperidone respectively.

### Example 18

### Pharmaceutical Tablets

Tablets containing risperidone monohydrochloride can be made according to the following guide:

Composition of the tablet mass per tablet, in mg

| | Strength(risperidone base) | | | | | | | |
|---|---|---|---|---|---|---|---|---|
| | 0.25 mg | 0.5 mg | 1 mg | 2 mg | 3 mg | 4 mg | 6 mg | 8 mg |
| Risperidone HCl (as base) | 0.25 | 0.5 | 1.0 | 2.0 | 3.0 | 4.0 | 6.0 | 8.0 |
| Lactose monohydrate* | 34.75 | 69.5 | 139.0 | 138.0 | 207.0 | 276.0 | 99.0 | 132.0 |
| Microcrystalline | 12.5 | 25.0 | 50.0 | 50.0 | 75.0 | 100.0 | 37.5 | 50.0 |
| cellulose | | | | | | | | |
| Sodium starch | 2.0 | 4.0 | 8.0 | 8.0 | 12.0 | 16.0 | 6.0 | 8.0 |
| glycollate | | | | | | | | |
| Mg stearate | 0.5 | 1.0 | 2.0 | 2.0 | 3.0 | 4.0 | 1.5 | 2.0 |
| TOTAL MASS | 50 | 100 | 200 | 200 | 300 | 400 | 150 | 200 |

The * indicates that the amount of lactose can be adjusted to compensate for (offset) the differing weights of the risperidone monohydrochloride salts. Optionally the tablets can be coated, as shown in the following table where the percentages refer to weight of the coating per the mass of the uncoated tablet.

| | Strength(risperidone base) | | | | | | | |
|---|---|---|---|---|---|---|---|---|
| | 0.25 mg | 0.5 mg | 1 mg | 2 mg | 3 mg | 4 mg | 6 mg | 8 mg |
| Opadry II | 5.0% | 3.25% | 2.0% | 2.0% | 1.83% | 1.65% | 2.25% | 2.0% |

Preparation of tablets:

Risperidone monohydrochloride salt, preferably the crystalline hemipentahydrate salt, is mixed well with 40% of the MCC (=10% tablet weight), then the other 60% of the MCC, and 30% of the lactose is added and mixed. The remaining lactose and the sodium starch glycollate are mixed, then Mg stearate is added and the blend is mixed, resulting in a blend for compression. The blend is compressed into tablets. The compressed tablets may be coated with the coating composition.

### EXAMPLE 19

### Pharmaceutical Tablets

Composition of the tablet mass per tablet, in mg

| | Strength (risperidone base) | | | | | | | |
|---|---|---|---|---|---|---|---|---|
| | 0.25 mg | 0.5 mg | 1 mg | 2 mg | 3 mg | 4 mg | 6 mg | 8 mg |
| Risperidone HCl.hph (as | 0.25 | 0.5 | 1.0 | 2.0 | 3.0 | 4.0 | 6.0 | 8.0 |
| base) | | | | | | | | |
| Lactose | 34.75 | 69.5 | 139.0 | 138.0 | 207.0 | 276.0 | 99.0 | 132.0 |
| monohydrate | | | | | | | | |
| Prosolv HD 90 | 2.5 | 25.0 | 50.0 | 50.0 | 75.0 | 100.0 | 37.5 | 50.0 |
| Sodium starch | 2.0 | 4.0 | 8.0 | 8.0 | 12.0 | 16.0 | 6.0 | 8.0 |
| glycollate | | | | | | | | |
| Mg stearate | 0.5 | 1.0 | 2.0 | 2.0 | 3.0 | 4.0 | 1.5 | 2.0 |
| TOTAL MASS | 50 | 100 | 200 | 200 | 300 | 400 | 150 | 200 |

Optional Coating

| | Strength(risperidone base) | | | | | | | |
|---|---|---|---|---|---|---|---|---|
| | 0.25 mg | 0.5 mg | 1 mg | 2 mg | 3 mg | 4 mg | 6 mg | 8 mg |
| Opadry II | 5.0% | 3.25% | 2.0% | 2.0% | 1.83% | 1.65% | 2.25% | 2% |

Preparation:

Crystalline monohydrochloride hemipentahydrate salt is mixed well with 10% of the Prosolv, then the 12.5% of the Prosolv is added and mixed. The remaining Prosolv (77.5%) is then added and mixed. 30% of the lactose and all of the sodium starch glycollate is added and mixed. Next 35% of the lactose is added and mixed. Finally the remaining lactose (35%) is added and mixed, then Mg stearate is added and the blend is mixed, resulting in a blend for compression. The blend is compressed into tablets containing. The compressed tablets may be coated with the coating composition.

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

We claim:

1. A monohydrochloride salt of risperidone.

2. The salt according to claim 1, wherein the ratio of risperidone ion to chloride ion is within the range of 0.8-1.2:1.

3. The salt according to claim 1, wherein the water solubility is less than 10 mg/ml.

4. The salt according to claim 3, wherein the water solubility is within the range of 5 to 9 mg/ml.

5. The salt according to claim 1 in crystalline form.

6. The salt according to claim 5, having a purity of at least 90%.

7. The salt according to claim 6, wherein said salt purity is at least 98%.

8. The salt according to claim 7, wherein said salt purity is at least 99%.

9. The salt according to claim 8, wherein said salt purity is at least 99.8%.

10. The salt according to claim 5, wherein said salt is a crystalline risperidone hydrochloride anhydrate.

11. The salt according to claim 10, which exhibits an x-ray powder diffraction pattern that substantially corresponds to FIG. 2.

12. The salt according to claim 5, wherein said salt is a hydrate having from about 7 to about 9.5% of water.

13. The salt according to claim 5, wherein said salt is crystalline risperidone hydrochloride hemipentahydrate.

14. The salt according to claim 13, which exhibits an x-ray powder diffraction pattern that substantially corresponds to FIG. 4.

15. A pharmaceutical composition comprising a risperidone monohydrochloride salt according to claim 1 and at least one pharmaceutically acceptable excipient.

16. The pharmaceutical composition according to claim 15, wherein said composition is a solid oral dosage and said risperidone salt is contained in an amount within the range of 0.1 to 20 mg, expressed in terms of the weight of risperidone base.

17. The pharmaceutical composition according to claim 16, wherein said risperidone salt is crystalline risperidone monohydrochloride hemipentahydrate.

18. The pharmaceutical composition according to claim 15, wherein said composition is a liquid dosage form that contains an effective anti-psychotic amount of said risperidone salt dissolved in a liquid excipient.

19. The pharmaceutical composition wherein said liquid excipient is water or a water and ethanol mixture.

20. The pharmaceutical composition according to claim 19, which further comprises sorbitol.

21. A process for making the salt according to claim 1, which comprises:
contacting a risperidone donor with a chloride ion donor in a solvent; and optionally
precipitating a crystalline risperidone monohydrochloride salt.

22. The process according to claim 21, wherein said risperidone donor is risperidone base or salt thereof; said chloride ion donor is hydrochloric acid or a chloride salt; and said solvent contains at least 10% water.

23. The process according to claim 21, wherein said risperidone donor is a risperidone salt of a weak acid; said chloride ion donor is a chloride salt; said solvent is at least 90% water; and said precipitating step forms crystalline risperidone hydrochloride hemipentahydrate.

24. The process according to claim 23, wherein said risperidone donor is risperidone acetate.

25. The process according to claim 21, wherein said solvent is water, ethanol or a mixture thereof.

26. A method for treating a psychotic disorder in a mammal, which comprises administering an effective anti-psychotic amount of the risperidone salt according to claim 1 to a mammal in need thereof.

27. A risperidone monohydrochloride hemipentahydrate.

28. A pharmaceutical composition comprising an effective anti-psychotic amount of a risperidone monohydrochloride according to claim 27 and at least one pharmaceutically acceptable excipient.

29. A risperidone monohydrochloride salt substantially free from a risperidone dihydrochloride salt.

30. The risperidone salt according to claim 29, wherein the amount of the risperidone dihydrochloride salt is not greater than 1% based on the total amount of risperidone salt.

31. A pharmaceutical composition comprising an effective anti-psychotic amount of the risperidone salt according to claim 30 and at least one pharmaceutically acceptable excipient.

32. The pharmaceutical composition according to claim 31, wherein said composition is a liquid dosage form.

33. The pharmaceutical composition according to claim 31, wherein said composition is a solid oral dosage form.
